(19) United States
(12) Patent Application Publication
(10) Pub. No.: US 2006/0275209 A1
(43) Pub. Date: Dec. 7, 2006

(54) Methods of [11c]-radiolabelling phenothiazine and phenothiazine-like compounds

(76) Inventors: Lutz F. Schweiger, Aberdeen (GB); Stuart A. Craib, Aberdeen (GB); Andrew E. Welch, Aberdeen (GB); Peter Sharp, Aberdeen (GB)

Correspondence Address: NIXON & VANDERHYE, PC, 901 NORTH GLEBE ROAD, 11TH FLOOR, ARLINGTON, VA, 22203, US

(21) Appl. No.: 10/573,882

(22) PCT Filed: Sep. 27, 2004
(86) PCT No.: PCT/GB04/04153

§ 371 Date: Jul. 19, 2006

(30) Foreign Application Priority Data
Sep. 29, 2003 (GB) 0322756.8

(51) Int. Cl. A61K 51/00; C07D 279/18

(52) U.S. Cl. 424/1.11; 544/37

(57) ABSTRACT

This invention pertains to methods of [¹¹C]-radiolabelling “phenothiazine” and “phenothiazine-like” compounds, which have a pendant group (which is a primary amino group; a cationic primary imino group; a secondary amino group; a cationic secondary imino group; a primary imino group; or a secondary imino group), by reaction with [¹¹C]methyl trifluoromethanesulfonate (CF₃SO₂O¹¹CH₃), also known as [¹¹C]methyl triflate. This reaction converts the pendant group into a [¹¹C]methyl-labelled pendant group. The resulting [¹¹C]-radiolabelling product is useful, for example, as an in vivo positron emission tomography (PET) tracer, for example, for patients suffering from melanoma, the most serious form of skin cancer, and tauopathy (e.g., Alzheimer's disease). The present invention also pertains to the resulting [¹¹C]-radiolabelling products, compositions comprising them, their use in methods of (e.g., PET) imaging, their use in methods of medical treatment and diagnosis, etc.

### RELATED APPLICATION

This application is related to United Kingdom patent application GB 0322756.8 filed 29 Sep. 2003, the contents of which are incorporated herein by reference in their entirety.

### TECHNICAL FIELD

This invention pertains generally to the field of radiochemical synthesis, and more specifically to methods of [¹¹C]-radiolabelling “phenothiazine” and “phenothiazine-like” compounds, which have a pendant group (which is a primary amino group; a cationic primary imino group; a secondary amino group; a cationic secondary imino group; a primary imino group; or a secondary imino group), by reaction with [¹¹C]methyl trifluoromethanesulfonate (CF₃SO₂O¹¹CH₃), also known as [¹¹C]methyl triflate. This reaction converts the pendant group into a [¹¹C]methyl-labelled pendant group. The resulting [¹¹C]-radiolabelling product is useful, for example, as an in vivo positron emission tomography (PET) tracer, for example, for patients suffering from melanoma, the most serious form of skin cancer, and tauopathy (e.g., Alzheimer's disease). The present invention also pertains to the resulting [¹¹C]-radiolabelling products, compositions comprising them, their use in methods of (e.g., PET) imaging, their use in methods of medical treatment and diagnosis, etc.

### BACKGROUND

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about”another particular value. When such a range is expressed, another embodiment includes from the one particular value and/or to the other particular value. Similarly, when values are expressed as approximations, by the use of the antecedent “about,” it will be understood that the particular value forms another embodiment.

Melanoma

Melanoma is the most serious form of skin cancer and claims around 2,000 lives each year in the United Kingdom of Great Britain (see, e.g., Cancer Research UK Website).

According to Cancer Research UK (see, e.g., Cancer Research UK Website) malignant melanoma is the 11th most common cancer in women, and the 12th most common cancer in men with over 5,700 new cases of melanoma each year in the UK.

Melanoma develops from cells producing melanin, a pigment that protects the deeper layers of the skin from the damaging effects of the sun:

Methylene Blue

Methylene blue (3,7-bis(dimethylamino)phenothiazine-5-ium chloride) is a low molecular weight, water soluble, tricyclic organic compound, which diffuses through the cellular membranes and accumulates selectively in melanoma cells (see, e.g., Link et a., 1998).

Methylene blue possesses a very high affinity to melanin by forming a charge transfer complex with the pigment (see, e.g., Potts, 1964).

Over several years, Link et al. have carried out clinical research focusing on methylene blue labelled with relatively long lived radioisotopes such as ²¹¹Astatine (²¹¹At, half-life (t_{1/2})=7.2 hours), ¹²³Iodine (¹²³I, t_{1/2}=13.2 hours) and ¹³¹Iodine (¹³¹I, t_{1/2}=8 days) (see, e.g., Link et al., 1998).

They investigated the α-particle emitter compound [²¹¹At]methylene blue as a therapeutic agent and were able to prove that this radioactive compound prevents metastatic spread and controls the growth of melanoma when given to human-melanoma-bearing animals (see, e.g., Link et al., 1998). They also investigated the γ-emitting ¹²³I— and the β-emitting [¹³¹]methylene blue compounds for diagnostic purposes of disseminated melanoma. Using a gamma camera, they concluded that in particular the ¹³¹I labelled compound was suitable for the detection of melanoma metastases (see, e.g., Link et al., 1998).

There is a great need for additional, and more powerful, radiolabelled phenothiazine and phenothiazine-like compounds, such as methylene blue.

The inventors have discovered novel methods for the fast and efficient synthesis of novel phenothiazine and phenothiazine-like compounds labelled with the short lived positron emitting ¹¹C isotope (t_{1/2}=20.4 minutes).

It is surprising and unexpected that the synthesis method is both fast (e.g., fast enough to compensate for the short half life), and efficient (e.g., efficient enough to provide sufficient radioactive yield to be useful).

¹¹C-labelled methylene blue is structurally identical to unlabelled methylene blue, and hence would show the same biodistribution, which is important for PET studies. Therefore [N-methyl-¹¹C]methylene blue is very useful, in particular as an in vivo PET tracer for patients suffering from melanoma, the most serious form of skin cancer, tauopathy (e.g., Alzheimer's disease), and other diseases.

### SUMMARY OF THE INVENTION

One aspect of the present invention pertains to a method of [¹¹C]-radiolabelling a phenothiazine compound or a phenothiazine-like compound, wherein:
said compound has a polycyclic core of three six-membered rings fused together in a linear fashion and denoted the A-ring, Bring, and C-ring, where the B-ring is the “middle” ring;
said polycyclic core is partially-aromatic or fully-aromatic;
said polycyclic core has 14 ring atoms, including exactly 1 or exactly 2 ring heteroatom(s), each of which is independently selected from N, O, and S;
the remainder of said ring atoms being C;
said exactly 1 or exactly 2 ring heteroatom(s) form part of the B-ring, but not part of the A-ring or C-ring, and so are located at one or both of the “central” positions denoted by a hash-mark (#) in the following depiction of the polycyclic core: 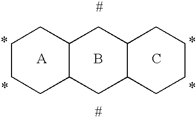
said compound has a pendant group covalently attached to a ring atom of said polycyclic core;
said pendant group is independently:
   a primary amino group;
   a cationic primary imino group;
   a secondary amino group;
   a cationic secondary imino group;
   a primary imino group; or
   a secondary imino group;
said method comprising the step of;
reacting said phenothiazine compound or a phenothiazine-like compound with [¹¹C]methyl trifluoromethanesulfonate (CF₃SO₂O¹¹CH₃);
thereby converting said pendant group to a corresponding [¹¹C]methyl-labelled pendant group, respectively:
   a [¹¹C]methyl-labelled secondary amino group;
   a [¹¹C]methyl-labelled cationic secondary imino group;
   a [¹¹C]methyl-labelled tertiary amino group;
   a [¹¹C]methyl-labelled cationic tertiary imino group;
   a [¹¹C]methyl-labelled secondary imino group; or
   a [¹¹C]methyl-labelled cationic tertiary imino group;
to give a [¹¹C]-radiolabelled phenothiazine or phenothiazine-like compound.

Another aspect of the invention pertains to a [¹¹C]-radiolabelled phenothiazine or phenothiazine-like compound, as described herein.

Another aspect of the invention pertains to a [¹¹C]-radiolabelled phenothiazine or phenothiazine-like compound which is obtained by, or obtainable by, a method as described herein.

Another aspect of the invention pertains to a composition (e.g., a pharmaceutical composition) comprising a [¹¹C]-radiolabelled phenothiazine or phenothiazine-like compound as described herein.

Another aspect of the invention pertains to a method of PET imaging which employs a [¹¹C]radiolabelled phenothiazine or phenothiazine-like compound as described herein.

Another aspect of the invention pertains to a [¹¹C]-radiolabelled phenothiazine or phenothiazine-like compound as described herein for use in a method of treatment of the human or animal body by therapy.

Another aspect of the invention pertains to use of a [¹¹C]-radiolabelled phenothiazine or phenothiazine-like compound as described herein in the manufacture of a medicament for use in the treatment of, e.g., skin cancer (e.g., melanoma) or a tauopathy (e.g., Alzheimer's disease).

Another aspect of the invention pertains to use of a method of [¹¹C]-radiolabelling a phenothiazine compound or a phenothiazine-like compound, as described herein, as part of a method of manufacturing a medicament for use in the treatment of, e.g., skin cancer (e.g., melanoma) a tauopathy (e.g., Alzheimer's disease).

Another aspect of the invention pertains to use of:
(i) an unlabelled phenothiazine compound or an unlabelled phenothiazine-like compound, wherein:
said compound has a polycyclic core of three six-membered rings fused together in a linear fashion and denoted the A-ring, B-ring, and C-ring, where the B-ring is the “middle” ring;
said polycyclic core is partially-aromatic or fully-aromatic;
said polycyclic core has 14 ring atoms, including exactly 1 or exactly 2 ring heteroatom(s), each of which is independently selected from N, O, and S;
the remainder of said ring atoms being C;
said exactly 1 or exactly 2 ring heteroatom(s) form part of the B-ring, but not part of the A-ring or C-ring, and so are located at one or both of the “central” positions denoted by a hash-mark (#) in the following depiction of the polycyclic core: 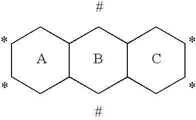
said compound has a pendant group covalently attached to a ring atom of said polycyclic core;
said pendant group is independently:
   a primary amino group;
   a cationic primary imino group;
   a secondary amino group;
   a cationic secondary imino group;
   a primary imino group; or
   a secondary imino group;
and
(ii) [¹¹C]methyl trifluoromethanesulfonate (CF₃SO₂O ¹¹CH₃);
in the manufacture of a medicament for use in the treatment of, e.g., skin cancer (e.g., melanoma) or a tauopathy (e.g., Alzheimer's disease).

Another aspect of the invention pertains to a method of treatment of, e.g., skin cancer (e.g., melanoma) or a tauopathy (e.g., Alzheimer's disease) in a patient, comprising administering to said patient a therapeutically-effective amount of a [¹¹C]-radiolabelled phenothiazine or phenothiazine-like compound as described herein.

Another aspect of the invention pertains to a [¹¹C]-radiolabelled phenothiazine or phenothiazine-like compound as described herein for use in a diagnostic or prognostic method practiced on the human or animal body.

Another aspect of the invention pertains to a method of diagnosis or prognosis (e.g., of skin cancer (e.g., melanoma) or a tauopathy (e.g., Alzheimer's disease)) which employs a [¹¹C]-radiolabelled phenothiazine or phenothiazine-like compound as described herein.

Another aspect of the invention pertains to use of a [¹¹C]-radiolabelled phenothiazine or phenothiazine-like compound as described herein in the manufacture of a medicament (e.g., a diagnostic or prognostic reagent) for use in diagnosis or prognosis, e.g., of skin cancer (e.g., melanoma) or a tauopathy (e.g., Alzheimer's disease).

Another aspect of the invention pertains to use of a [¹¹C]-radiolabelled phenothiazine or phenothiazine-like compound as described herein, as part of a method of manufacturing a medicament (e.g., a diagnostic or prognostic reagent) for use in diagnosis or prognosis e.g., of skin cancer (e.g., melanoma) or a tauopathy (e.g., Alzheimer's disease).

Another aspect of the invention pertains to use of:
(i) a phenothiazine compound or a phenothiazine-like compound, wherein:
said compound has a polycyclic core of three six-membered rings fused together in a linear fashion and denoted the A-ring, B-ring, and C-ring, where the B-ring is the “middle” ring;
said polycyclic core is partially-aromatic or fully-aromatic;
said polycyclic core has 14 ring atoms, including exactly 1 or exactly 2 ring heteroatom(s), each of which is independently selected from N, O, and S;
the remainder of said ring atoms being C;
said exactly 1 or exactly 2 ring heteroatom(s) form part of the B-ring, but not part of the A-ring or C-ring, and so are located at one or both of the “central” positions denoted by a hash-mark (#) in the following depiction of the polycyclic core: 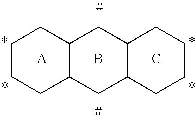
said compound has a pendant group covalently attached to a ring atom of said polycyclic core;
said pendant group is independently:
   a primary amino group;
   a cationic primary imino group;
   a secondary amino group;
   a cationic secondary imino group;
   a primary imino group; or
   secondary imino group;
and
(ii) [¹¹C]methyl trifluoromethanesulfonate (CF₃SO₂O ¹¹CH₃);
in the manufacture of a medicament (e.g., a diagnostic or prognostic reagent) for use in diagnosis or prognosis, e.g., of skin cancer (e.g., melanoma) or a tauopathy (e.g., Alzheimer's disease).

As will be appreciated by one of skill in the art, features and preferred embodiments of one aspect of the invention will also pertain to other aspects of the invention.

### BRIEF DESCRIPTION OF THE DRAWINGS

FIG. 1 is (a) a radioactivity-chromatogram of [N-methyl-¹¹C]methylene blue (98%, 7.8 minutes) (the minor peak at 5.8 minutes is unidentified) and (b) a UV-chromatogram of non radioactive methylene blue (7.8 minutes).

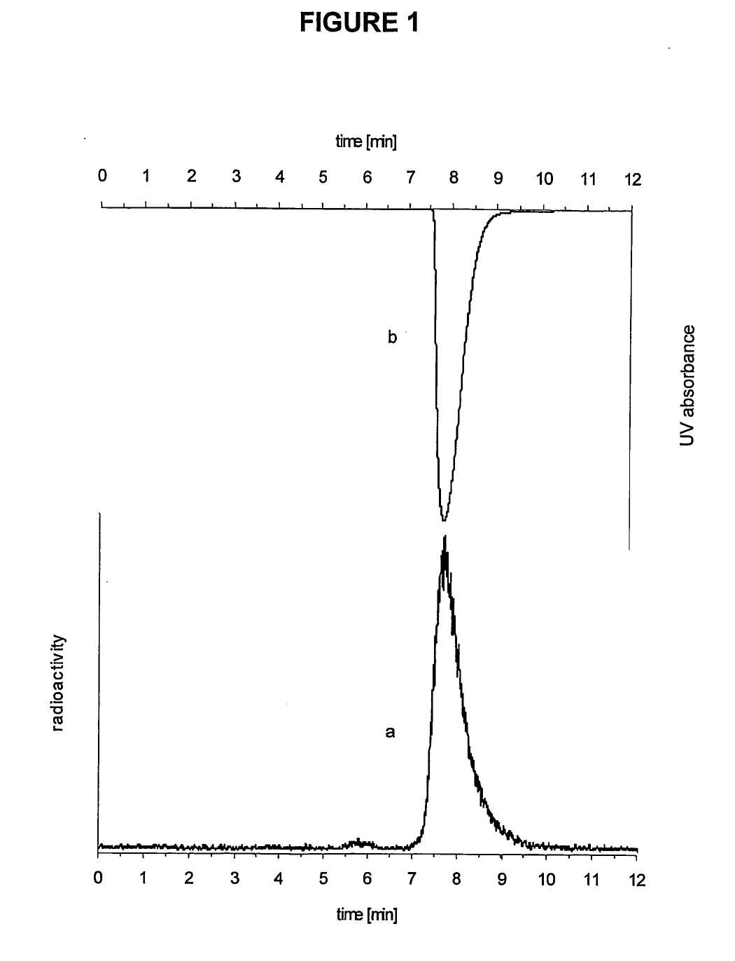

### DETAILED DESCRIPTION OF THE INVENTION

The present invention pertains to both to methods of [¹¹C]-radiolabelling certain compounds, and the resulting [¹¹C]-radiolabelled compounds.

One aspect of the present invention pertains to methods of [¹¹C]-radiolabelling “phenothiazine” and “phenothiazine-like” compounds, which have a pendant group which is independently:
a primary amino group;
a cationic primary imino group;
a secondary amino group;
a cationic secondary imino group;
a primary imino group; or
a secondary imino group;
   by reaction with [¹¹C]methyl trifluoromethanesulfonate (CF₃SO₂O¹¹CH₃), also known as [¹¹C]methyl triflate. This reaction (i.e., ¹¹C-methylation) converts the pendant group into a corresponding [¹¹C]methyl-labelled pendant group, respectively:
a [¹¹C]methyl-labelled secondary amino group;
a [¹¹C]methyl-labelled cationic secondary imino group;
a [¹¹C]methyl-labelled tertiary amino group;
a [¹¹C]methyl-labelled cationic tertiary imino group;
a [¹¹C]methyl-labelled secondary imino group; or
a [¹¹C]methyl-labelled cationic tertiary imino group.

An especially preferred embodiment of novel methods of the present invention is a method of [¹¹C]-radiolabelling Azure B (a “phenothiazine” compound having a pendant secondary amino group; an example of an (unlabelled) phenothiazine compound or (unlabelled) phenothiazine-like compound) to produce [N-methyl-¹¹C]methylene blue, by reaction with the [¹¹]methyl trifluoromethanesulfonate. In a further preferred embodiment, the reaction is performed in the presence of K₂CO₃ in H₂O, as shown, for example, in the following scheme.

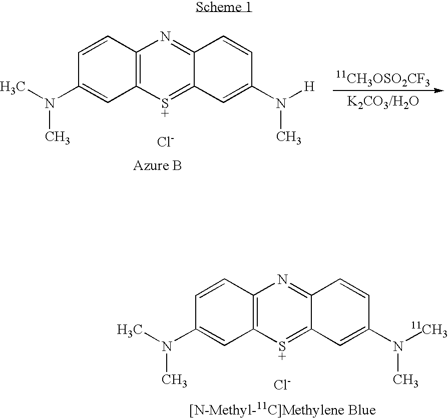

Reagents, Reaction Conditions, and Purification

In one embodiment, the reaction is performed in the presence of a suitable Bronsted base. Examples of suitable Bronsted bases include, but are not limited to carbonates and bicarbonates, e.g., alkali metal carbonates and bicarbonate, e.g., sodium and potassium carbonates and bicarbonate, e.g., potassium carbonate (K₂CO₃).

In one embodiment, the reaction is carried out in aqueous media. For example, in one embodiment, the [¹¹C]methyl triflate is introduced into an aqueous solution (or suspension) of the phenothiazine or phenothiazine-like compound and (optionally) a suitable Bronsted base, e.g., potassium carbonate (K₂CO₃), to form a reaction mixture.

In one embodiment, the reaction mixture (of [¹¹C]methyl triflate; phenothiazine or phenothiazine-like compound; and optionally Bronsted base) is mixed (e.g., stirred), e.g., for a mixing (e.g., stirring) time of about 1-30 minutes (e.g., about 1-10 minutes; e.g., about 5 minutes).

In one embodiment, the reaction is carried out at ambient or room temperature (e.g., 20° C.-25° C.).

In one embodiment, the reaction is carried out under an inert atmosphere (e.g., argon).

For example, an argon filled vial equipped with a magnetic stirring bar is filled with a solution of phenothiazine or phenothiazine-like compound and K₂CO₃ in sterile water and subsequently placed on a magnetic stirrer 5 minutes prior to end of bombardment (EOB). [¹¹C]methyl triflate is then trapped in the purple solution. The trapped amount usually reaches a maximum (on average 2.6 GBq) after 15 minutes (from EOB). The magnetic stirrer is then switched on and the solution stirred for 5 minutes at room temperature (e.g., 20° C.-25° C.) resulting in the [¹¹C]methylation of the phenothiazine or phenothiazine-like compound with [¹¹C]methyl triflate.

In one embodiment, the resulting [¹¹C]-radiolabelled product is purified using ion exchange methods, e.g., with ion exchange media, e.g., using cation exchange methods, e.g., with cation exchange media, e.g., a cation exchange cartridge, e.g., a small disposable cation exchange cartridge.

For example, the reaction mixture may be transferred to a cation exchange cartridge (immobilising the [¹¹C]-radiolabelled product), which is then washed, e.g., with ethanol and sterile water. Washing removes not only unreacted starting material but also up to 98% of the radioactive [¹¹C]by-products. The cartridge is then eluted, e.g., with sodium chloride solution, e.g., sterile 0.9% w/v sodium chloride solution, to release the [¹¹C]-radiolabelled product.

The synthesis (and optionally purification) may readily be performed very quickly, e.g., in less than 60 minutes, e.g., in less than 45 minutes, e.g., in less than 40 minutes, e.g., in less than 35 minutes, e.g., in 10-60 minutes, e.g., in 10-45 minutes, e.g., in 10-40 minutes, e.g., in 10-35 minutes, e.g., in 15-60 minutes, e.g., in 15-45 minutes, e.g., in 15-40 minutes, e.g., in 15-35 minutes, e.g., in 20-60 minutes, e.g., in 20-45 minutes, e.g., in 20-40 minutes, e.g., in 20-35 minutes; from the end of bombardment (EOB).

It is anticipated that synthesis yield and product purity can be further improved by optimisation, for example, optimisation of the bombard time and intensity, reaction solvents, reaction conditions (e.g., temperature), etc.

Radiochemical purity and specific activity of the [¹¹C]-radiolabelled product (solution) may be determined using, for example, HPLC.

The identity of the [¹¹C]-radiolabelled product may be confirmed, for example, by co-injection with the corresponding unlabelled product, and noting that the retention time is identical for both.

In one embodiment, the method provides a radiochemical purity greater than 90%, preferably greater than 95%, preferably greater than 96%, preferably greater than 97%.

In one embodiment, the method provides a radiochemical yield of at least 2%, preferably at least 3%, preferably at least 4%, e.g., 4-10%, e.g., 4-6%.

In one embodiment, the method provides a product with a specific average activity of at least 0.5 GBq/μmol, preferably at least 1.0 GBq/μmol, preferably at least 1.5 GBq/μmol.

Phenothiazine and Phenothiazine-Like Compounds

The present invention pertains to methods of [¹¹C]-radiolabelling “phenothiazine” and “phenothiazine-like” compounds.

Such compounds are characterized by a polycyclic core of three six-membered rings fused together in a linear fashion, said polycyclic core having 14 ring atoms, including exactly 1 or exactly 2 ring heteroatom(s), each of which is independently selected from nitrogen, oxygen, and sulfur; and remainder of the ring atoms being C. More specifically, one of the ring atoms is independently N, O, or S; another of the ring atoms is independently C, N, O, or S; and the remainder of the ring atoms is C. No other rings are fused to the polycyclic core.

In one embodiment, said polycyclic core has 14 ring atoms, including exactly 1 ring heteroatom selected from nitrogen, oxygen, and sulfur; and the remainder of the rings atoms is C. More specifically, one of the ring atoms is independently N, O, or S; and the remainder of the ring atoms is C.

In one embodiment, said polycyclic core has 14 ring atoms, including exactly 2 ring heteroatoms selected from nitrogen, oxygen, and sulfur; and the remainder of the rings atoms is C. More specifically, one of the ring atoms is independently N, O, or S; another of the ring atoms is independently N, O, or S; and the remainder of the ring atoms is C.

The three six-membered rings are fused together in a linear fashion, and denoted the A-ring, B-ring, and C-ring, where the B-ring is the “middle” ring, as shown in the following depiction of the polycyclic core.

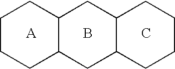

The exactly 1 or exactly 2 ring heteroatom(s) form part of the B-ring, but not part of the A-ring or C-ring, and so are located at one or both of the “central” positions denoted by a hash-mark (#) in the following depiction of the polycyclic core.

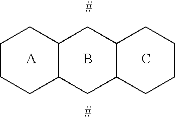

In one embodiment, the core has exactly 1 ring heteroatom.

In one embodiment, the core has exactly 1 ring heteroatom which is independently selected from O, N, and S.

In one embodiment, the core has exactly 1 ring heteroatom which is independently selected from O and N.

In one embodiment, the core has exactly 1 ring heteroatom: O.

In one embodiment, the core has exactly 1 ring heteroatom: N.

In one embodiment, the core has exactly 1 ring heteroatom: S.

In one embodiment, the core has exactly 2 ring heteroatoms.

In one embodiment, the core has exactly 2 ring heteroatoms, each of which is independently selected from O, N and S.

In one embodiment, the core has exactly 2 ring heteroatoms, each of which is independently selected from N and S.

In one embodiment, the core has exactly 2 ring heteroatoms: N and S.

In one embodiment, the core has exactly 2 ring heteroatoms: N and O.

In one embodiment, the core has exactly 2 ring heteroatoms: N and N.

In one embodiment, the core has exactly 2 ring heteroatoms: O and O.

In one embodiment, the core has exactly 2 ring heteroatoms: O and S.

In one embodiment, the core has exactly 2 ring heteroatoms: S and S.

The polycyclic core is partially-aromatic (i.e., not all of the ring atoms contribute to the aromatic character of the polycyclic core), or fully-aromatic (i.e., all of the ring atoms contribute to the aromatic character of the polycyclic core). In one embodiment, the polycyclic core is fully-aromatic.

In one especially preferred embodiment, the exactly 1 or 2 heteroatom(s) are N and S (and are referred to herein as “phenothiazine” compounds):

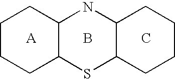

An example of such a polycyclic core is found in phenothiazine:

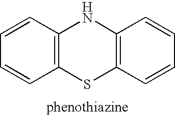

In other embodiments, the exactly 1 or 2 heteroatom(s) are as defined herein, but are other than N and S (and are referred to herein as “phenothiazine-like” compounds).

Examples of such polycyclic cores are found in the following compounds:

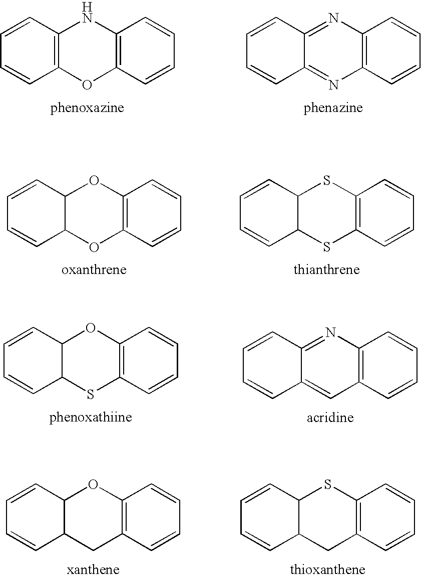

The Pendant Group

The phenothiazine and phenothiazine-like compounds have a pendant group which is independently:
a primary amino group;
a cationic primary imino group;
a secondary amino group;
a cationic secondary imino group;
a primary imino group; or
a secondary imino group.

The term “pendant group,” as used herein, pertains to a group which is covalently attached to a ring atom of the polycyclic core of the phenothiazine compound or phenothiazine-like compound. For example, the pendant group does not form part of a ring of the polycyclic core of (i.e., is not fused to) the phenothiazine compound or phenothiazine-like compound.

A pendant primary amino group is a group of the formula —NH₂.

A pendant cationic primary imino group is ═N(⁺)H₂.

A pendant secondary amino group is a group of the formula —NHR.

A pendant cationic secondary imino group is ═N(⁺)HR.

A pendant primary imino group is a group of the formula ═NH.

A pendant secondary imino group is a group of the formula ═NR.

Thus, in one embodiment, the pendant group is independently selected from:

—NH₂, —NHR, ═N(⁺)H₂, ═N(⁺)HR, ═NH, and ═NR.

In one embodiment, the pendant group is independently a secondary amino group or a cationic secondary imino group.

In one embodiment, the pendant group is independently selected from:

—NHR and ═N(⁺)HR.

The [¹¹C]Methyl Radiolabelled Pendant Group

Upon reaction with the radiolabelled methylating agent, [¹¹C]methyl trifluoromethanesulfonate (CF₃SO₂O¹¹CH₃; [¹¹C]methyl triflate), the pendant group is converted to the corresponding [¹¹C]methyl-labelled pendant group.

Thus, the [¹¹C]methyl-radiolabelled phenothiazine and phenothiazine-like compounds have a pendant group which is independently:
a [¹¹C]methyl-labelled secondary amino group;
a [¹¹C]methyl-labelled cationic secondary imino group;
a [¹¹C]methyl-labelled tertiary amino group;
a [¹¹C]methyl-labelled cationic tertiary imino group;
a [¹¹C]methyl-labelled secondary imino group; or
a [¹¹C]methyl-labelled cationic tertiary imino group.

A pendant primary amino group (—NH₂) gives rise to a corresponding [¹¹C]methyl-labelled secondary amino group: —NH—(¹¹CH₃).

A cationic primary imino group (═N(⁺)H₂) gives rise to a corresponding [¹¹C]methyl-labelled cationic secondary imino group: ═N(⁺)H—(¹¹CH₃).

A pendant secondary amino group (—NHR) gives rise to a corresponding [¹¹C]methyl-labelled tertiary amino group: —NR—(¹¹CH₃).

A cationic secondary imino group (═N(⁺)HR) gives rise to a corresponding [¹¹C]methyl-labelled cationic tertiary imino group: ═N(⁺)R—(¹¹CH₃).

A pendant primary imino group (═NH) gives rise to a corresponding [¹¹C]methyl-labelled secondary imino group: ═N—(¹¹CH₃).

A pendant secondary imino group (═NR) gives rise to a corresponding [¹¹C]methyl-labelled cationic tertiary imino group: ═N(⁺)R—(¹¹CH₃).

The conversion of the pendant group to the corresponding [¹¹C]methyl-labelled pendant group is summarised in the following table.

TABLE 1

| | | Corresponding [¹¹C]Methyl-Labelled | |
|---|---|---|---|
| Pendant Group | | Pendant Group | |
| primary amino | —NH₂ | —NH—(¹¹CH₃) | [¹¹C]methyl-labelled secondary |
| group | | | amino group |
| cationic primary | ═N⁽⁺⁾H₂ | ═N⁽⁺⁾H—(¹¹CH₃) | [¹¹C]methyl-labelled cationic |
| imino group | | | secondary imino group |
| secondary amino | —NHR | —NR—(¹¹CH₃) | [¹¹C]methyl-labelled tertiary |
| group | | | amino group |
| cationic secondary | ═N⁽⁺⁾HR | ═N⁽⁺⁾R—(¹¹CH₃) | [¹¹C]methyl-labelled cationic |
| imino group | | | tertiary imino group |
| primary imino group | ═NH | ═N—(¹¹CH₃) | [¹¹C]methyl-labelled secondary |
| | | | imino group |
| secondary imino | ═NR | ═N⁽⁺⁾R—(¹¹CH₃) | [¹¹C]methyl-labelled cationic |
| group | | | tertiary imino group |

Thus, in one embodiment, the [¹¹C]methyl-labelled pendant group is independently selected from: —NH—(¹¹CH₃), —NR—(¹¹CH₃), ═N(⁺)H—(¹¹CH₃), ═N(⁺)R—(¹¹CH₃), and ═N—(¹¹CH₃).

In one embodiment, the [¹¹C]methyl-labelled pendant group is independently a secondary amino group, or a corresponding cationic imino group.

In one embodiment, the [¹¹C]methyl-labelled pendant group is independently selected from: —NR—(¹¹CH₃) and ═N(⁺)R—(¹¹CH₃).

The Pendant Group: Position

In one embodiment, the pendant group is independently attached to a ring carbon atom of the polycyclic core of the phenothiazine or phenothiazine-like compound.

In one embodiment, the pendant group is independently attached to a ring carbon atom of the A-ring or C-ring of the polycyclic core of the phenothiazine or phenothiazine-like compound.

In one embodiment, the pendant group is independently attached to a ring carbon atom of the A-ring or C-ring, but not of the B-ring, of the polycyclic core of the phenothiazine or phenothiazine-like compound.

In one embodiment, the pendant group is independently attached at one of the “distal” positions of the A-ring or C-ring of the polycyclic core of the phenothiazine or phenothiazine-like compound, which positions are denoted by asterisks (*) in the following depiction of the polycyclic core:

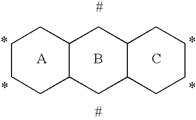

The Pendant Group: The Substituent R

In one embodiment, R is independently selected from: C₁₋₆alkyl, C₁₋₆alkenyl, C₁₋₆alkynyl, C₁₋₆cycloalkyl, and C₁₋₆cycloalkenyl, and is optionally substituted with one or more (e.g., 1, 2, 3, 4, etc.) groups selected from halo (e.g., fluoro, chloro, bromo, iodo), hydroxy, and C₁₋₄alkoxy.

In one embodiment, R is independently C₁₋₆alkyl.

In one embodiment, R is independently C₁₋₄alkyl.

In one embodiment, R is independently -Me, -Et, -nPr, -iPr, -nBu, -iBu, -sBu, or -tBu.

In one embodiment, R is independently -Me or -Et.

In one embodiment, R is independently -Et.

In one embodiment, R is independently -Me.

Additional Substituents

In addition to the pendant group discussed above, the phenothiazine or phenothiazine-like compound optionally has one or more (e.g., 1, 2, 3, 4, etc.) additional substituents, for example, selected from: amino (—NH₂), methylamino (—NHMe), dimethylamino (—NMe₂), ethylamino (—NHEt), diethylamino (—NEt₂), imino (═NH), methylimino (═NMe), ethylimino (═NEt), methyl (-Me), ethyl (-Et), fluoro (—F), chloro (—Cl), bromo (—Br), iodo (—I), oxo (═O), hydroxy (—OH), carboxy (—COOH), and protonated and deprotonated forms thereof.

Ionic, Salt, and Solvate Forms

In addition, the phenothiazine or phenothiazine-like compound may be in any ionic (e.g., with a suitable counter-ion), salt (e.g., acid addition salt, e.g., hydrochloride salt), or solvate (e.g., hydrate) form.

For example, an amino group (—NH₂) may be in the form of an HCl addition salt: —NH₂.HCl (or —N(⁺)H₃Cl⁻).

Some Preferred Phenothiazine Compounds

In one embodiment, the phenothiazine or phenothiazine-like compound is a compound of the following formula: 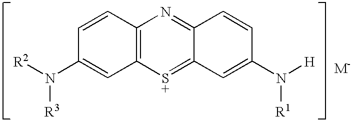
wherein each of R¹, R², and R³ is independently —H or as defined above for R; and M⁻ is an anion (e.g., to achieve electrical neutrality).

In one embodiment, R¹ is independently as defined above for R.

In one embodiment, —NHR¹ is independently —NHMe.

In one embodiment, —NR²R³ is independently —NH₂.

In one embodiment, —NR²R³ is independently —NHMe.

In one embodiment, —NR²R³ is independently —NMe₂.

In one embodiment, M⁻ is independently a halide ion.

In one embodiment, M⁻ is independently F—, Cl⁻, Br⁻, or I⁻.

In one embodiment, M⁻ is independently Cl⁻, Br⁻, or I⁻.

In one embodiment, M⁻ is independently Cl⁻.

In one embodiment, M⁻ is independently Br⁻.

In one embodiment, M⁻ is independently I⁻.

In one especially preferred embodiment, phenothiazine or phenothiazine-like compound is Azure B (wherein —NHR¹ is —NHMe; —NR²R³ is —NMe₂; and M⁻ is Cl⁻). 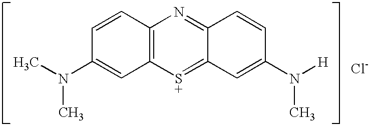
and the resulting [¹¹C]-radiolabelled phenothiazine or phenothiazine-like compound is [N-methyl-¹¹]methylene blue:

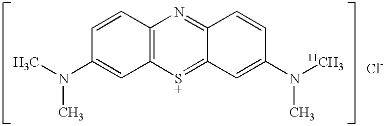

Resonance Structures

It is noted that may chemical moieties and compounds have resonance properties. Such species may be considered to alternate or resonate between two or more resonance structures. Any of these different resonance structures may be used to accurately represent the species. Usually, but not without exception, the most energetically-stable resonance is used to depict the species. As will be appreciated by the skilled artisan, the structures shown herein are often one of many possible resonance structures which may be drawn to depict the same compound. As used herein, and unless otherwise specified, a reference to one structure is to be considered a reference to all possible corresponding resonance structures.

For example, there are many resonance structures for Azure B, including the ones shown below. Each of these equivalently represents the same compound.

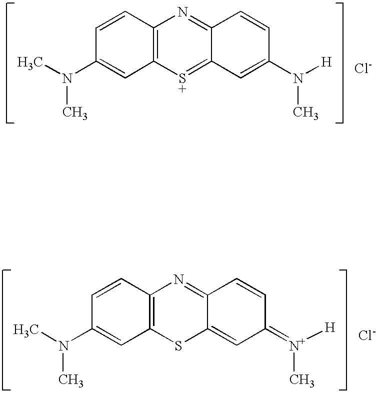

Some Specific Examples

Some specific examples of phenothiazine and phenothiazine-like compounds include, but are not limited to, the following:

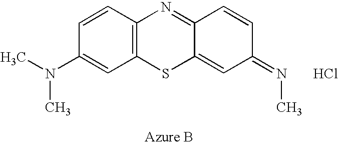

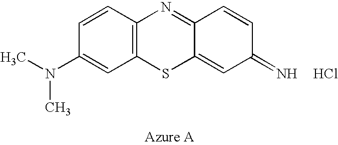

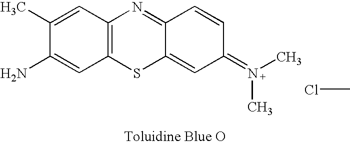

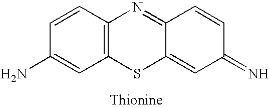

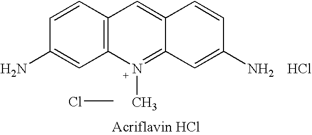

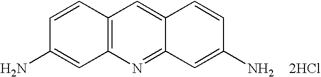

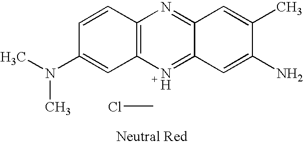

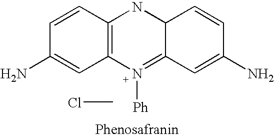

Preparation of Radiolabelled Methylating Reagent: [¹¹C]Methyl Triflate

The methods of the present invention employ the methylating reagent [¹¹C]methyl trifluoromethanesulfonate (CF₃S(═O)₂O—¹¹CH₃), also known as [¹¹C]methyl triflate.

It is noted that [¹¹C]methyl iodide is not only the fastest reacting methyl halide in nucleophilic substitution (S_{N}2) reactions such as N—, O — and S-methylation procedures (see, e.g., Bolton, 2001), but it is also regarded as the most commonly used labelling agent for the preparation of ¹¹C-radiotracers (see, e.g., Nagren et al., 1995). However, efforts to use [¹¹C]methyl iodide as the methylating agent with Azure B have proven unsatisfactory, providing very low radioactive yield and radiochemical purity: the highest radiochemical yield was less than 0.5%.

[¹¹C]Methyl triflate has been used in radioactive labelling reactions (see, e.g., Bolton, 2001; Jewett, 1992; Iwata et al., 2001; Nagren et al., 1995; Lundkvist et al., 1998; Nagren et al., 1998). None of these publications teach or suggest the use of [¹¹C]methyl triflate in the methods described herein.

As demonstrated herein, use of [¹¹C]methyl triflate as a methylating agent greatly increased not only the radioactive yield but also the radiochemical purity.

[¹¹C]Methyl triflate may be prepared, for example, using the methods discussed below.

In a first step (“irradiation”), a mixture of nitrogen and oxygen, at high pressure (e.g., about 1-5 MPa, e.g., about 2 MPa) is subjected to bombardment with high energy (e.g., about 5-20 MeV, e.g., about 10 MeV) protons to form ¹¹CO₂ via a ¹⁴N(p,α)¹¹C nuclear reaction. A beam current of about 10-100 μA (e.g., about 30 μA) and an irradiation time of about 1-120 minutes (e.g., about 10 minutes) is suitable.

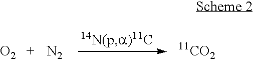

In a second step (“methoxide formation”), the resulting ¹¹CO₂ is reduced to form ¹¹CH₃O⁻, using a suitable reducing agent, for example, lithium aluminium hydride (LiAlH₄, LAH). See, for example, Crouzel et al., 1987. At the “end of bombardment” (EOB), ¹¹CO₂ is transferred, for example, in a stream of helium gas, into a solution of LAH, for example, a cooled 0.1 M solution of LAH in tetrahydrofuran (THF). The ¹¹CO₂ reacts with LAH to produce the ¹¹CH₃O⁻. The solvent (e.g., THF) may be removed by heating, for example, to 130° C.

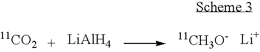

In a third step (“neutralisation”), the resulting ¹¹CH₃O⁻ is neutralised to form the corresponding alcohol, ¹¹CH₃OH, using, for example, a Bronsted acid, for example, phosphoric acid. For example, after removal of solvent, the ¹¹CH₃O⁻ is cooled, for example, to 0° C., phosphoric acid (e.g., 1 ml of 10% phosphoric acid) is added.

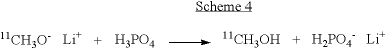

In a fourth step (“iodination”), the resulting ¹¹CH₃OH is then reacted with hydroiodic acid (HI). For example, the ¹¹CH₃OH is transferred, e.g., distilled, to another reaction containing HI, and, for example, heated, for example, to 100-150° C. (e.g., 135° C.) to produce ¹¹CH₃I.

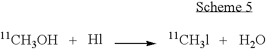

In a fifth step (“triflate formation”), the resulting ¹¹CH₃I is then reacted with a suitable triflate salt, for example silver triflate (AgCF₃SO₃). The reaction may conveniently be performed using column methods, for example, using a column packed with silver triflate. See, for example, Jewett, 1992. For example, a suitable column (e.g., stainless steel HPLC C-18 Luna column (250×3 mm)) is loosely packed with coarse silver triflate, and held in place with, for example, glass wool. Before use, the column is suitably conditioned, for example, under argon gas flow for 30 minutes at 300° C. The ¹¹CH₃I, in a steam of carrier gas, for example, helium gas, is then passed through the column which is heated to a suitable temperature, for example, about 100-300° C. (e.g., about 200° C.), to yield the desired CF₃S(═O)₂O —¹¹CH₃.

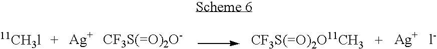

In one embodiment, the methods of [¹¹C]-radiolabelling a phenothiazine compound or a phenothiazine-like compound further comprise the earlier step of (5) triflate formation.

In one embodiment, the methods further comprise the earlier step of (4) iodination and (5) triflate formation.

In one embodiment, the methods further comprise the earlier step of (3) neutralisation, (4) iodination, and (5) triflate formation.

In one embodiment, the methods further comprise the earlier step of (2) methoxide formation, (3) neutralisation, (4) iodination, and (5) triflate formation.

In one embodiment, the methods further comprise the earlier step of (1) irradiation, (2) methoxide formation, (3) neutralisation, (4) iodination, and (5) triflate formation.

Automation

In one embodiment, the method of [¹¹C]-radiolabelling a phenothiazine compound or a phenothiazine-like compound is partially or fully automated.

In one embodiment, the method is fully automated.

The method may be automated using well known apparatus and techniques.

[¹¹C]-Radiolabelled Phenothiazine and Phenothiazine-Like Compounds

One aspect of the present invention pertains to [¹¹C]-radiolabelled phenothiazine and phenothiazine-like compounds which are obtained by, or are obtainable by, a method as described herein.

One aspect of the present invention pertains to [¹¹C]-radiolabelled phenothiazine and phenothiazine-like compounds, as described herein.

In one embodiment, the compound is a [¹¹C]-radiolabelled phenothiazine compound having the following formula wherein R¹, R², R³, and M⁻ is as defined herein:

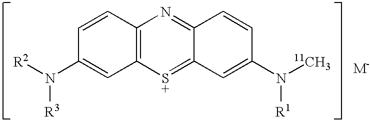

In one embodiment, —NHR¹ is independently —NHMe.

In one embodiment, —NR²R³ is independently —NH₂.

In one embodiment, —NR²R³ is independently —NHMe.

In one embodiment, —NR²R³ is independently —NMe₂.

In one embodiment, M⁻ is independently a halide ion.

In one embodiment, M⁻ is independently Cl⁻.

In an especially preferred embodiment, the compound is [N-methyl-¹¹C]methylene blue:

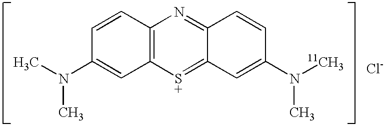

Compositions

One aspect of the present invention pertains to compositions comprising a [¹¹C]-radiolabelled phenothiazine and phenothiazine-like compound, as described herein.

One aspect of the present invention pertains to compositions comprising a [¹¹C]-radiolabelled phenothiazine and phenothiazine-like compound which is obtained by, or is obtainable by, a method as described herein.

In one embodiment, the composition further comprises a pharmaceutically acceptable carrier.

Methods of Imaging

One aspect of the present invention pertains to methods of (e.g., PET) imaging which employ a [¹¹C]-radiolabelled phenothiazine or phenothiazine-like compound, as described herein.

One aspect of the present invention pertains to methods of (e.g., PET) imaging which employ a [¹¹C]-radiolabelled phenothiazine or phenothiazine-like compound which is obtained by, or is obtainable by, a method as described herein.

One aspect of the present invention pertains to methods of (e.g., PET) imaging which includes, as additional prior steps, the steps of a method of [¹¹C]-radiolabelling a phenothiazine or phenothiazine-like compound, as described herein.

In one embodiment, the methods of imaging comprise the following steps:
(i) introducing the [¹¹C]-radiolabelled phenothiazine or phenothiazine-like compound into a subject;
(ii) imaging (e.g., a part of, the whole of) the subject.

In one embodiment, the step of (ii) imaging the subject is the step of (ii) determining the presence and/or location and/or amount of [¹¹C]-radiolabelled phenothiazine or phenothiazine-like compound in (e.g., a part of, the whole of) the subject.

Methods of PET imaging are well known. See, for example, Czernin et al., 2002; Goh et al., 2003; Van Heertum et al., 2003; Fowler et al., 1999; Kennedy et al., 1997.

For example, in one embodiment, the method is a method of PET imaging comprising the steps of:
(i) preparing a [¹¹C]-radiolabelled phenothiazine or phenothiazine-like compound using a method according to any one of claims 1 to 45;
(ii) introducing said compound into a subject; and
(iii) PET imaging (e.g., a part of, the whole of) the subject.

Methods of Medical Treatment

One aspect of the present invention pertains to a [¹¹C]-radiolabelled phenothiazine or phenothiazine-like compound, as described herein, for use in a method of treatment (e.g., of a disease condition) of the human or animal body by therapy.

One aspect of the present invention pertains to a [¹¹C]-radiolabelled phenothiazine or phenothiazine-like compound, which is obtained by, or is obtainable by, a method as described herein, for use in a method of treatment (e.g., of a disease condition) of the human or animal body by therapy.

One aspect of the present invention pertains to use of a [¹¹C]-radiolabelled phenothiazine or phenothiazine-like compound, as described herein, in the manufacture of a medicament for use in the treatment of a disease condition.

One aspect of the present invention pertains to use of a [¹¹C]-radiolabelled phenothiazine or phenothiazine-like compound, which is obtained by, or is obtainable by, a method as described herein, in the manufacture of a medicament for use in the treatment of a disease condition.

One aspect of the present invention pertains to use of a method of [¹¹C]-radiolabelling a phenothiazine or a phenothiazine-like compound, as described herein, as part of a method of manufacturing a medicament for use in the treatment of a disease condition.

One aspect of the present invention pertains to a method of manufacturing a medicament for use in the treatment of a disease condition which includes the steps of [¹¹C]-radiolabelling a phenothiazine or a phenothiazine-like compound, as described herein.

One aspect of the present invention pertains to use of:
(i) a (unlabelled) phenothiazine compound or a (unabelled) phenothiazine-like compound, as described herein; and
(ii) [¹¹C]methyl trifluoromethanesulfonate (CF₃SO₂O¹¹CH₃); in the manufacture of a medicament for use in the treatment of a disease condition.

One aspect of the present invention pertains to a method of treatment of a disease condition in a patient, comprising administering to said patient a therapeutically-effective amount of a [¹¹C]-radiolabelled phenothiazine or phenothiazine-like compound, as described herein.

One aspect of the present invention pertains to a method of treatment of a disease condition in a patient, comprising administering to said patient a therapeutically-effective amount of a [¹¹C]-radiolabelled phenothiazine or phenothiazine-like compound, which is obtained by, or is obtainable by, a method as described herein.

One aspect of the present invention pertains to a method of treatment of a disease condition in a patient, comprising administering to said patient a therapeutically-effective amount of a [¹¹C]-radiolabelled phenothiazine or phenothiazine-like compound, and which includes, as additional prior steps, the steps of a method of [¹¹C]-radiolabelling a phenothiazine or phenothiazine-like compound, as described herein.

In one embodiment, the disease condition is skin cancer.

In one embodiment, the disease condition is melanoma.

In one embodiment, the disease condition is a tauopathy.

In one embodiment, the disease condition is Alzheimer's disease (AD).

Tauopathy

As discussed in Wischik et al 2002, labelled phenothiazine and phenothiazine-like compounds of the type described herein can bind to “Paired Helical Filaments” (PHFs) and can serve as ligands for tau aggregates.

Such compounds may therefore be used in methods of labelling aggregated PHF tau, for example, for the purpose of diagnosis or prognosis of a tauopathy, such as Alzheimer's Disease (AD).

Notably, it is not only Alzheimer's Disease in which tau protein (and aberrant function or processing thereof) may play a role. The pathogenesis of neurodegenerative disorders such as Pick's disease and Progressive Supranuclear Palsy (PSP) appears to correlate with an accumulation of pathological truncated tau aggregates in the dentate gyrus and stellate pyramidal cells of the neocortex, respectively. Other dementias include fronto-temporal dementia (FTD); parkinsonism linked to chromosome 17 (FTDP-17); disinhibition-dementia-parkinsonism-amyotrophy complex (DDPAC); pallido-ponto-nigral degeneration (PPND); Guam-ALS syndrome; pallido-nigro-luysian degeneration (PNLD); cortico-basal degeneration (CBD) and others (see, e.g., Wischik et al., 2000, especially Table 5.1 therein). Each of these diseases, which is characterized primarily or partially by abnormal tau aggregation, is referred to herein as a “tauopathy.”

In particular, the compounds may be used to assess neurofibrillary degeneration associated with tauopathies, e.g., in a subject who may be believed to suffer from any of the above-mentioned diseases.

Methods of Diagnosis or Prognosis

One aspect of the present invention pertains to a [¹¹C]-radiolabelled phenothiazine or phenothiazine-like compound, as described herein, for use in a diagnostic or prognostic method (e.g., of a disease condition) practiced on the human or animal body.

One aspect of the present invention pertains to a [¹¹C]-radiolabelled phenothiazine or phenothiazine-like compound, which is obtained by, or obtainable by, a method described herein, for use in a diagnostic or prognostic method (e.g., diagnosis or prognosis of a disease condition) practiced on the human or animal body.

One aspect of the present invention pertains to a method of diagnosis or prognosis (e.g., of a disease condition) which employs a [¹¹C]-radiolabelled phenothiazine or phenothiazine-like compound, as described herein.

One aspect of the present invention pertains to a method of diagnosis or prognosis (e.g., of a disease condition) which employs a [¹¹C]-radiolabelled phenothiazine or phenothiazine-like compound, which is obtained by, or obtainable by, a method described herein.

One aspect of the present invention pertains to a method of diagnosis or prognosis (e.g., of a disease condition) which employs a [¹¹C]-radiolabelled phenothiazine or phenothiazine-like compound, and which includes, as additional prior steps, the steps of a method of [¹¹C]-radiolabelling a phenothiazine or phenothiazine-like compound, as described herein.

One aspect of the present invention pertains to use of a [¹¹C]-radiolabelled phenothiazine or phenothiazine-like compound, as described herein, in the manufacture of a medicament (e.g., a diagnostic or prognostic reagent) for use in the diagnosis or prognosis of a disease condition.

One aspect of the present invention pertains to use of a [¹¹C]-radiolabelled phenothiazine or phenothiazine-like compound, which is obtained by, or obtainable by, a method described herein, in the manufacture of a medicament (e.g., a diagnostic or prognostic reagent) for use in the diagnosis or prognosis of a disease condition.

One aspect of the present invention pertains to use of a method of [¹¹C]-radiolabelling a phenothiazine or a phenothiazine-like compound, as described herein, as part of a method of preparing a diagnostic or prognostic reagent suitable for use in a method of diagnosis or prognosis (e.g., of a disease condition).

One aspect of the present invention pertains to a method of manufacturing a medicament for use in the diagnosis or prognosis (e.g., of a disease condition) which includes the steps of [¹¹C]-radiolabelling a phenothiazine or a phenothiazine-like compound, as described herein.

One aspect of the present invention pertains to use of:
(i) a (unlabelled) phenothiazine compound or a (unabelled) phenothiazine-like compound, as described herein; and
(ii) [¹¹C]methyl trifluoromethanesulfonate (CF₃SO₂O¹¹CH₃);
   in the manufacture of (e.g., in a method of preparing) a medicament (e.g., a diagnostic or prognostic reagent) for use in the diagnosis or prognosis of a disease condition.

In one embodiment, the disease condition is a tauopathy.

In one embodiment, the disease condition is Alzheimer's disease (AD).

In one embodiment, the diagnostic or prognostic method is determining the AD state of a subject.

In one embodiment, the method of diagnosis or prognosis includes, as additional prior steps, the steps of a method of [¹¹C]-radiolabelling a phenothiazine or phenothiazine-like compound, as described herein.

In one embodiment, the methods of diagnosis or prognosis comprise the following steps:
(i) introducing the [¹¹C]-radiolabelled phenothiazine or phenothiazine-like compound into the subject;
(ii) determining the presence and/or location and/or amount of [¹¹C]-radiolabelled phenothiazine or phenothiazine-like compound in the subject;
(iii) correlating the result of the determination made in (ii) with a disease condition of the subject.

In one embodiment, the methods of [¹¹C]-radiolabelling phenothiazine or phenothiazine-like compounds, as described herein, are followed by the additional steps of:
(i) introducing the [¹¹C]-radiolabelled phenothiazine or phenothiazine-like compound into a subject;
(ii) determining the presence and/or location and/or amount of [¹¹C]-radiolabelled phenothiazine or phenothiazine-like compound in the subject;
(iii) correlating the result of the determination made in (ii) with a disease condition of the subject.

For example, in one embodiment, the methods of diagnosis or prognosis of a tauopathy comprise the following steps:
(i) introducing the [¹¹C]-radiolabelled phenothiazine or phenothiazine-like compound into the subject;
(ii) determining the presence and/or amount of [¹¹C]-radiolabelled phenothiazine or phenothiazine-like compound bound to aggregated PHF tau in the brain of the subject;
(iii) correlating the result of the determination made in (ii) with the tauopathy (e.g., AD) state of the subject.

In one embodiment, the methods of [¹¹C]-radiolabelling phenothiazine or phenothiazine-like compounds, as described herein, are followed by the additional steps of:
(i) introducing the [¹¹C]-radiolabelled phenothiazine or phenothiazine-like compound into a subject;
(ii) determining the presence and/or amount of [¹¹C]-radiolabelled phenothiazine or phenothiazine-like compound bound to aggregated PHF tau in the brain of the subject;
(iii) correlating the result of the determination made in (ii) with the tauopathy (e.g., AD) state of the subject.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

The term “treatment” includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

Routes of Administration

The [¹¹C]-radiolabelled phenothiazine or phenothiazine-like compound, or pharmaceutical composition comprising it, may be administered to a subject/patient by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal (including, e.g., intracatheter injection into the brain); by implant of a depot or reservoir, for example, subcutaneously or intramuscularly.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

Formulations

While it is possible for the [¹¹C]-radiolabelled phenothiazine or phenothiazine-like compound to be used (e.g., administered) alone, it is often preferable to present it as a formulation.

One aspect of the present invention pertains to compositions comprising a [¹¹C]-radiolabelled phenothiazine and phenothiazine-like compound which is obtained by, or is obtainable by, a method as described herein, and a carrier.

One aspect of the present invention pertains to compositions comprising a [¹¹C]-radiolabelled phenothiazine and phenothiazine-like compound, as described herein, and a carrier.

In one embodiment, the composition is a pharmaceutical composition (e.g., formulation, preparation, medicament) comprising a compound, as described herein, and a pharmaceutically acceptable carrier.

In one embodiment, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and 1. Ash), 2001 (Synapse Information Resources, Inc., Endicott, New York, USA), Remington's Pharmaceutical Sciences, 20th edition, pub. Lippincoft, Williams & Wilkins, 2000; and Handbook of Pharmaceutical Excipients, 2nd edition, 1994.

Another aspect of the present invention pertains to methods of making a pharmaceutical composition comprising admixing at least one [¹¹C]-radiolabelled phenothiazine or phenothiazine-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier+which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the active compounds, and compositions comprising the active compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

### EXAMPLES

The following are examples are provided solely to illustrate the present invention and are not intended to limit the scope of the invention, as described herein.

Chemicals and Solvents

All reagents were purchased from Sigma-Aldrich and used without further purification unless otherwise noted. All used solvents were purified and degassed according to standard procedures.

Analytical Methods

All analyses of the labelled compounds were performed with a Gynkothek HPLC system (P580 pump) and variable Wavelength UV/VIS detector (at 664 nm) coupled in series with a BIOSCAN Nal detector (B-FC-3200). The HPLC system was operated using a Phenomenex Luna C-18 column (150×3.0 mm, particle size: 5 μm). The eluent was produced by adding 0.75% of acetic acid and 0.25% of methane sulfonic acid to a mixture of HPLC grade acetonitrile and distilled water (1:4). The eluent was filtered and degassed with helium before use. The flow rate was set at 1 ml/min.

Preparation of Silver Trifluoromethanesulfonate Column

A silver trifluoromethanesulfonate (silver triflate) column was prepared according to the method described by Jewett, 1992. Coarse silver triflate (1.0 g) and Graphpac-GC 80/100 (2.0 g, Alltech) was ground to a homogenous mixture. An empty stainless steel HPLC C-18 Luna column (250×3 mm) was loosely packed (10 cm length) with the mixture in the central region, and to restrain the packing material, both ends of the column were then fitted with glass wool. Before the first reaction, the column was inserted into a tube furnace (Carbolite furnaces) and conditioned under argon gas flow for 30 minutes at 300° C.

[¹¹C]Carbon Dioxide Radiosynthesis

[¹¹C]Carbon dioxide was prepared by proton bombardment of a gas mixture (98% N₂, 2% O₂) by the ¹⁴N(p,α)¹¹C nuclear reaction. The gas target was pressurised to 270 psi (1.9 MPa) and irradiated with 11 MeV protons produced by the CTI RDS-111 cyclotron at the John Mallard Scottish P.E.T. Centre in Aberdeen, Scotland. Irradiations of 10 minutes with a beam current of 27 μA were typically used.

[¹¹C]Methyl Iodide Radiosynthesis

[¹¹C]Methyl iodide was prepared according to the traditional lithium aluminium hydride (LAH)/hydroiodic acid (HI) method (see, for example, Crouzel et al., 1987). At the “end of bombardment” (EOB), [¹¹C]carbon dioxide was transferred from the target in a stream of helium gas to the remote controlled automated [¹¹C]methyl iodide module, where it was passed into 200 μl of a cooled 0.1 M solution of LAH in tetrahydrofuran (THF). The [¹¹C]carbon dioxide reacted with LAH to produce the [¹¹C]methoxide anion. The first reaction vessel was then heated to 130° C. to evaporate the solvent. After completing the THF evaporation, the contents of the reaction vessel were cooled to 0° C. and 1 ml of 10% phosphoric acid was added to synthesise [¹¹C]methanol. [¹¹C]Methanol was then distilled into the second reaction vessel containing 600 μl of hydroiodic acid (HI). The second reaction vessel was heated to 135° C. to produce on average 4.8 GBq of [¹¹C]methyl iodide. The average specific activity was 780 GBq/mmol.

[¹¹C]Methyl Trifluoromethanesulfonate Radiosynthesis

[¹¹C]Methyl trifluoromethanesulfonate ([¹¹C]methyl triflate) was prepared according to the method described by Jewett, 1992. In a stream of helium gas, the [¹¹C]methyl iodide was passed through the silver triflate graphpac column which was connected in series to the [¹¹C]methyl iodide module. The column was inserted into a tube furnace operated at 200° C., synthesising on average 2.0 GBq of [¹¹C]methyl triflate.

[N-methyl-¹¹C]Methylene Blue Radiosynthesis

[N-methyl-¹¹C]methylene blue was prepared from Azure B using [¹¹C]methyl triflate. The [¹¹C]methyl triflate was trapped in a reaction vessel containing a solution of Azure B (1 mg, 3.27 μmol) and potassium carbonate (K₂CO₃) (20 mg, 144.72 μmol) in 1.5 mL of sterile water. After the collection of [¹¹C]methyl triflate, the solution was stirred at room temperature (RT, 20° C. ) for 5 minutes.

The solution was transferred on to a cation exchange cartridge (Waters, Sep-Pak Accell Plus CM) which was washed with 5 ml of ethanol and 15 ml of sterile water. Then the cartridge was eluted with 10 ml of sterile 0.9% w/v sodium chloride solution to yield [N-methyl-¹¹C]methylene blue. Radiochemical purity and specific activity of the final solution was determined by HPLC.

The identity of the radiolabelled product was confirmed via co-injection with a commercial sample of methylene blue. The retention time in the UV-chromatogram was identical to the retention time of [N-methyl-¹¹C]methylene blue in the radioactivity-chromatogram.

In all cases, [N-methyl-¹¹C]methylene blue was obtained with a radiochemical purity greater than 97% in an averaged 4-6% radiochemical yield based on [¹¹C]methyl iodide. The average specific activity was 1.5 G Bq/μmol.

Analytical HPLC showed the product to be >97% radiochemically pure in a 4-6% radiochemical yield and to co-elute with a commercial sample of methylene blue at the same retention time of 7.8 minutes (see FIG. 2).

On average, only 7-10 μg/ml of Azure B could be found in the product rinse, as determined by the UV detection spectrum.

The total synthesis time from EOB was 35 minutes.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention as defined by the appended claims.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Bolton R, 2001, “Isotopic methylation,” J. Labelled Comp. Radiopharm., Vol. 44, pp. 701-736.
Cancer Research UK Website. http://www.cancerresearchuk.org/aboutcancer/specific cancers/15216.
Crouzel C, L\dot{a}angström B, Pike V W, Coenen H, 1987, “Recommendations for a practical production of [¹¹C]methyl iodide,” Appl. Radiat. Isot., Vol. 38, pp. 601-603.
Czernin J, et al., 2002, “Positron emission tomography scanning: Current and future applications,” Annual Review of Medicine, Vol. 53, pp. 89-112.
Fowler J S, et al., 1999, “PET and drug research and development,” Journal of Nuclear Medicine, Vol. 40, No. 7, pp.1154-1163.
Goh A S W et al., 2003, “Clinical positron emission tomography imaging—Current applications,” Annals Academy of Medicine Singapore, Vol. 32, No. 4, pp. 507-517.
Iwata R, Pascali C, Bogni A, Miyake Y, Yanai K, Ido T, 2001, “A simple loop method for the automated preparation of [¹¹C]raclopride from [¹¹C]methyl triflate,” Appl. Radiat. Isot., Vol. 55, pp.17-22.
Jewett D M, 1992, “A simple synthesis of [¹¹C]methyl triflate,” Appl. Rad. Isot., Vol. 43, pp. 1383-1385.
Kennedy S H, et al.,1997, “A review of functional neuroimaging in mood disorders: Positron emission tomography and depression,” Canadian Journal of Psychiatry-Revue Canadienne de Psychiatrie, Vol. 42, No. 5, pp. 467-475.
Link E M, Blower P J, Costa D C, Lane D M, Lui D, Brown R S D, Eli P J, Spittle M F, 1998, “Early detection of melanoma metastases with radioiodinated methylene blue,” Eur. J. Nucl. Med., Vol. 25, pp. 1322-1329.
Lundkvist C, Sandell J, N\dot{a}gren K, Pike V W, Halldin C, 1998, “Improved synthesis of PET radioligands, [¹¹C]FLB 457, [¹¹C]MDL and [¹¹C]β—CIT-FE, by the use of [¹¹C]methyl triflate,” J. Labelled Comp. Radiopharm., Vol.41, pp. 545-556.
N\dot{a}gren K, Halldin C, 1998, “Methylation of amide and thiol functions with [¹¹C]methyl triflate, as exemplified by [¹¹C]NMSP, [¹¹C]flumazenil and [¹¹C]methionine,” J. Labelled Comp. Radiopharm., Vol. 41, pp. 831-841.
N\dot{a}gren K, Müller L, Halldin C, Swahn C G, Lehikoinen P, 1995, “Improved synthesis of some commonly used PET radioligands by the use of [¹¹C]methyl triflate,” Nucl. Med. Biol., Vol.22, pp. 235-239.
Potts A M, 1964, “The reaction of uveal pigment with polycyclic compounds,” Invest. Opthmol. Visual. Sci., Vol. 3, pp.405-416.
Van Heertum R L, et al., 2003, “Positron emission tomography and single-photon emission computed tomography brain imaging in the evaluation of dementia,” Seminars in Nuclear Medicine, Vol. 33, No. 1, pp. 77-85.
Wischik et al., 2000, “Neurobiology of Alzheimer's Disease”, in The Molecular and Cellular Neurobiology Series, Eds. Dawbarn et al., (Bios Scientific Publishers, Oxford).
Wischik et al., 2002, “Neurofibrillary Labels,” published International (PCT) Patent Application publication No. WO 02/075318, published 26 Sep. 2002.

1. A method of [¹¹C]-radiolabelling a phenothiazine compound or a phenothiazine-like compound, wherein:
said compound has a polycyclic core of three six-membered rings fused together in a linear fashion and denoted the A-ring, B-ring, and C-ring, where the B-ring is the “middle” ring;
said polycyclic core is partially-aromatic or fully-aromatic;
said polycyclic core has 14 ring atoms, including exactly 1 or exactly 2 ring heteroatom(s), each of which is independently selected from N, O, and S;
the remainder of said ring atoms being C;
said exactly 1 or exactly 2 ring heteroatom(s) form part of the B-ring, but not part of the A-ring or C-ring, and so are located at one or both of the “central” positions denoted by a hash-mark (#) in the following depiction of the polycyclic core: 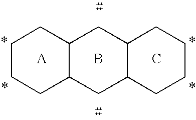
said compound has a pendant group covalently attached to a ring atom of said polycyclic core;
said pendant group is independently:
a primary amino group;
a cationic primary imino group;
a secondary amino group;
a cationic secondary imino group;
a primary imino group; or
a secondary imino group;
said method comprising the step of:
reacting said phenothiazine compound or a phenothiazine-like compound with [¹¹C]methyl trifluoromethanesulfonate (CF₃SO₂O¹¹CH₃);
thereby converting said pendant group to a corresponding [¹¹C]methyl-labelled pendant group, respectively:
a [¹¹C]methyl-labelled secondary amino group;
a [¹¹C]methyl-labelled cationic secondary imino group;
a [¹¹C]methyl-labelled tertiary amino group;
a [¹¹C]methyl-labelled cationic tertiary imino group;
a [¹¹C]methyl-labelled secondary imino group; or
a [¹¹C]methyl-labelled cationic tertiary imino group;
to give a [¹¹C]-radiolabelled phenothiazine or phenothiazine-like compound.

2-62. (canceled)
